(19) United States
(12) Patent Application Publication
(10) Pub. No.: US 2019/0137464 A1
(43) Pub. Date: May 9, 2019

(54) K-9 CONTRABAND INSPECTION SEAT

(71) Applicants: Robert Henry Kasper, Carmel, NY (US); Michael Brown Kasper, Holmes, NY (US); Daniel Kevin Styczynski, Whitehall, NY (US)

(72) Inventors: Robert Henry Kasper, Carmel, NY (US); Michael Brown Kasper, Holmes, NY (US); Daniel Kevin Styczynski, Whitehall, NY (US)

(21) Appl. No.: 16/182,923

(22) Filed: Nov. 7, 2018

Related U.S. Application Data
(60) Provisional application No. 62/582,494, filed on Nov. 7, 2017.

(51) Int. Cl. G01N 33/00; A47C 7/74

(52) U.S. Cl. CPC G01N 33/0001; A47C 7/744

(57) ABSTRACT

An inspection seat includes a seat back that defines a rear aperture. A set of seat sides includes a seat left side attached to the seat back and a seat right side attached to the seat back. A seat top is attached to one or the other of the set of seat sides and the seat back. The seat top defines a top aperture. The inspection seat still further includes a seat front attached to the set of seat sides. The seat top, the set of seat sides, and the seat front define a lower volume below the seat top. The rear aperture enables air flow communication between a space exterior to the lower volume and the lower volume. The top aperture enables air flow communication between a space above the seat top and the lower volume.

### CROSS-REFERENCE TO RELATED APPLICATIONS

This application claims priority to U.S. Provisional Patent Application No. 62/582,494, filed on Nov. 7, 2017, the entire disclosure of which is hereby incorporated by reference.

### BACKGROUND OF THE DISCLOSURE

### 1. Field of the Disclosure

The present disclosure relates to devices configured to aid detection of contraband on persons and objects while more specifically relating to seats configured to separate trained contraband detection animals from human subjects while providing a partially enclosed space from which the animal can draw inhalation.

### 2. Discussion of the Prior Art

In past disclosures, canines or other animals have been disclosed to search for contraband such as drugs or explosives. This is because animals have noses that are far more sensitive and responsive to a wide variety of materials than humans. Dogs, for example, are known to be able to detect various materials in the parts per billion range. Canines have been used particularly because they can be trained to respond in a predetermined manner when they detect the presence of a target substance.

In the modern world, it is becoming more and more important to be able to detect drugs, explosives and other contraband that produces a detectable scent. This is particularly true at airport security check points, national border entry points and other key locations where people and vehicles need to be checked.

However, simply having a dog sniff for substances at a check point is a procedure that may annoy or frighten an innocent person, or it might even be an unacceptable invasion of privacy. This is particularly true in a place like an airport security check point where numerous people of all ages, genders, nationalities, and religions routinely pass through. This problem might even arise with persons passing through a check point if the substance was hidden on their body high enough to put it above the dog's smelling range.

It is known that many prohibited materials, such as explosives and drugs emit scents which are distinctive and can be detected in very small quantities by dogs which have been trained to identify such target scents. However, bringing a dog into direct contact with a large number of pedestrians or vehicles presents difficulties as well. Some people are fearful of dogs and others may cause harm to a highly trained dog or its handler. Traveling among a large number of vehicles may also create the potential for injury to a dog and/or its handler. As such, improvements are desired.

### SUMMARY

This summary is provided to introduce a selection of concepts in a simplified form that are further described below in the detailed description. This summary is not intended to identify key factors or essential features of the claimed subject matter, nor is it intended to be used to limit the scope of the claimed subject matter.

In an example, an inspection seat includes a seat back that defines a rear aperture. The inspection seat also includes a set of seat sides. The set of seat sides includes a seat left side attached to the seat back and a seat right side attached to the seat back. The inspection seat further includes a seat top attached to one or the other of the set of seat sides and the seat back. The seat top defines a top aperture. The inspection seat still further includes a seat front attached to the set of seat sides. The seat top, the set of seat sides, and the seat front define a lower volume below the seat top. The rear aperture enables air flow communication between a space exterior to the lower volume and the lower volume. The top aperture enables air flow communication between a space above the seat top and the lower volume.

In an example, a method of locating contraband includes providing an inspection seat. The inspection seat includes a seat back that defines a rear aperture. The inspection seat also includes a set of seat sides. The set of seat sides includes a seat left side attached to the seat back and a seat right side attached to the seat back. The inspection seat further includes a seat top attached to one or the other of the set of seat sides and the seat back. The seat top defines a top aperture. The inspection seat still further includes a seat front attached to the set of seat sides. The seat top, the set of seat sides, and the seat front define a lower volume below the seat top. The rear aperture enables air flow communication between a space exterior to the lower volume and the lower volume. The top aperture enables air flow communication between a space above the seat top and the lower volume. The method also includes seating an object on the inspection seat. The method further includes promoting an air flow containing a scent from the space above the seat top to the lower volume. The method still further includes detecting contraband objects by analysis of the scent of the air flow.

The following description and annexed drawings set forth certain illustrative aspects and implementations. These are indicative of but a few of the various ways in which one or more aspects may be employed. Other aspects, advantages, and/or novel features of the disclosure will become apparent from the following detailed description when considered in conjunction with the annexed drawings.

### DESCRIPTION OF THE DRAWINGS

FIG. 1 is a perspective view illustration of an example inspection seat according to at least one embodiment of the present disclosure;

FIG. 2 is an exploded view of the inspection seat of FIG. 1;

FIG. 3 is a detail view of a seat back of the inspection seat of FIG. 1;

FIG. 4 is a detail view of a seat right side of the inspection seat of FIG. 1;

FIG. 5 is a detail view of a seat front of the inspection seat of FIG. 1;

FIG. 6 is a detail view of a seat top of the inspection seat of FIG. 1;

FIG. 7 is a detail view of a seat key of the inspection seat of FIG. 1;

FIG. 8 is a detail view of a rear mounting bar of the inspection seat of FIG. 1;

FIG. 9 is a front view of the seat back of the inspection seat of FIG. 1;

FIG. 10 is a front view of a partial assembly of the inspection seat of FIG. 1, showing a seat right side attached to the seat back;

FIG. 11 is a detail view of a front mounting bar of the inspection seat of FIG. 1;

FIG. 12 is a front view of a partial assembly of the inspection seat of FIG. 1, showing the seat right side and a seat left side attached to the seat back;

FIG. 13 is a front view of a partial assembly of the inspection seat of FIG. 1, showing the seat right side, the seat left side, and the seat front attached to the seat back;

FIG. 14 is a front view of a fully assembled inspection seat of FIG. 1;

FIG. 15 is an illustration of a disassembled inspection seat of FIG. 1, showing the relatively small volume required to store/transport the inspection seat; and

FIG. 16 is a top-down flow chart illustrating a method of using the inspection seat of FIG. 1.

### DETAILED DESCRIPTION

The claimed subject matter is now described with reference to the drawings, wherein like reference numerals are used to refer to like elements throughout. In the following description, for purposes of explanation, numerous specific details are set forth in order to provide an understanding of the claimed subject matter. It is evident, however, that the claimed subject matter may be practiced without these specific details. In other instances, structures and devices are illustrated in block diagram form in order to facilitate describing the claimed subject matter. Relative size, orientation, etc. of parts, components, etc. may differ from that which is illustrated while not falling outside of the scope of the claimed subject matter. For the purposes of this disclosure, the term “analysis of the scent of the air flow” can include analysis of elemental, molecular, and compound presence within a volume of air. This analysis can be done through an animal olfactory and neural network, or through automated means be they mechanical, chemical, etc.

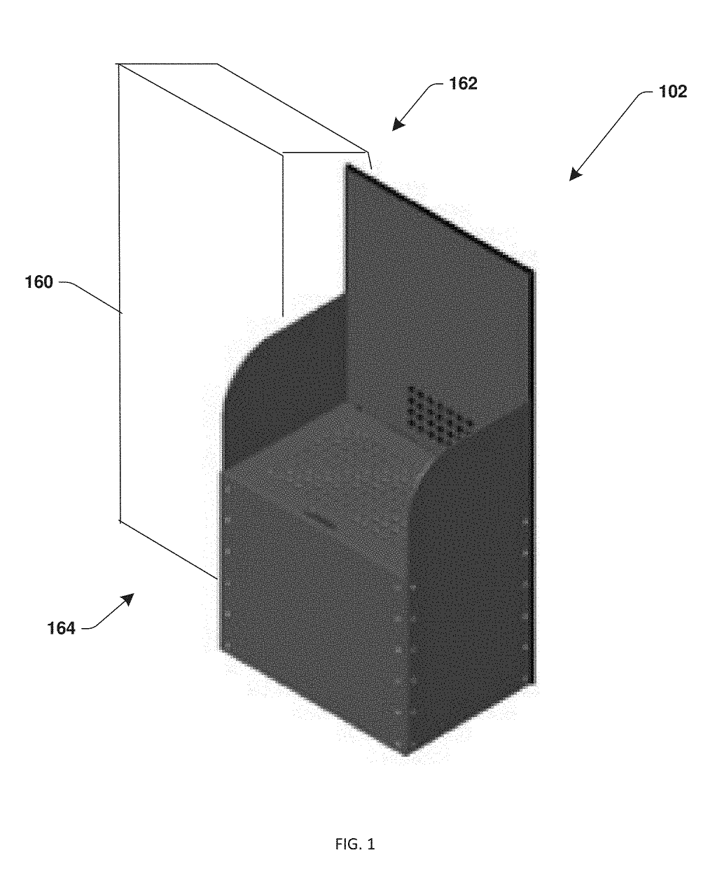

Referring to FIG. 1, a perspective view of an assembled inspection seat 102 is shown. The inspection seat 102 can also be termed a contraband detection chair or any other numerous names. Regardless of the naming convention the inspection seat 102 provides a less intrusive or a non-intrusive way to ease detection of contraband on people through the use of scent detection. In an example, the scent detection is provided by a detection trained animal, such as a canine. The inspection seat 102 can be a part of a less intrusive process to help prevent people from bringing illegal contraband into correctional facilities and may also provide more probable cause for a law enforcement agency to strip, frisk, and search a person suspected of concealing or possessing contraband. However, it will be appreciated that the inspection seat 102 is not limited to these types of events, and in some examples, may be used at other locations and types of special events such as concerts, sporting events, etc.

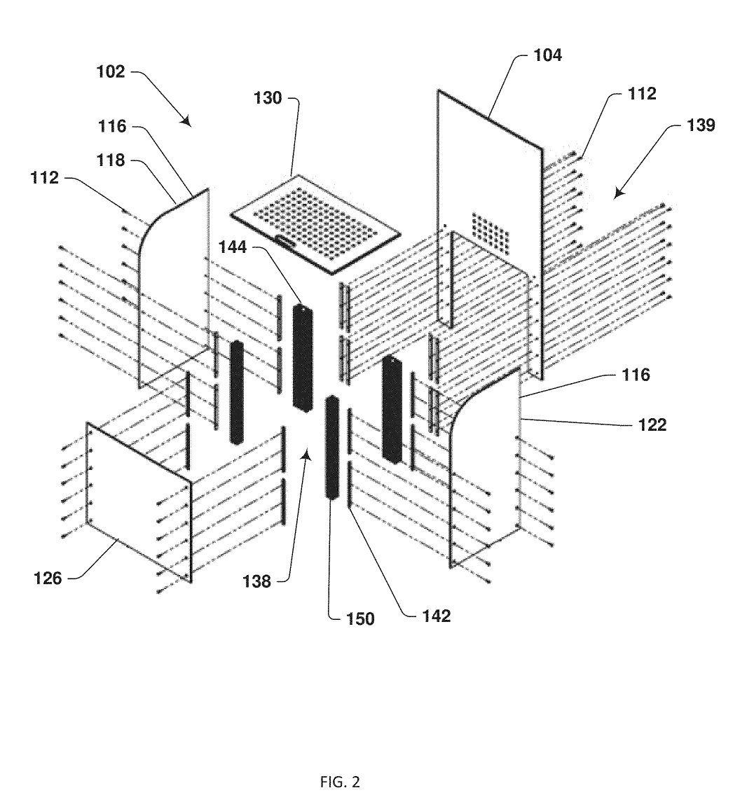

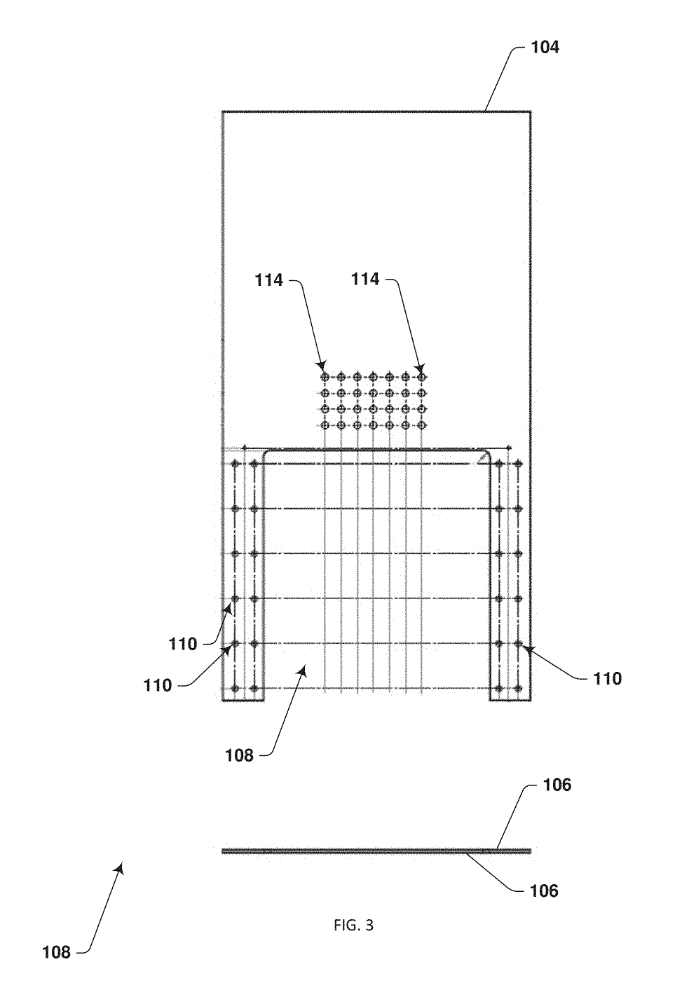

Referring to FIG. 2, an exploded view of the inspection seat 102 is shown. The inspection seat 102 includes a seat back 104. Referring to FIG. 3, a detail view of the seat back 104 is shown. The seat back 104 can be constructed of any suitable material that can meet the expected performance characteristics of the inspection seat 102. In an example, the seat back 104 comprises an aluminum alloy (e.g., aluminum 6061). In an example, the seat back 104 comprises an aluminum alloy with a coating 106 (e.g., a black powder coating, an epoxy powder coating, etc.).

As shown in FIG. 3, the seat back 104 defines a rear aperture 108. In an example, such as the one shown, the seat back 104 defines the rear aperture 108 with three sides (e.g., a right side, a left side, and a top side) with the fourth side being a surface such as a floor upon which the inspection seat 102. Of course, other examples are anticipated, such as a seat back that defines the rear aperture 108 such that the rear aperture 108 is closed on all sides of the rear aperture 108.

In an example, the rear aperture 108 has dimensions of about 19½-inches high (up and down in the figure) by about 17½-inches wide (left-to-right in the figure). The dimensions of the rear aperture 108 are selected and manufactured such that an animal (e.g., a canine) can put its head or a portion of its head through the rear aperture 108 as will be further discussed below.

The seat back 104 can also define several through holes 110 to facilitate mounting structure that will be further described below. In an example, the several through holes 110 can also include countersunk portions to enable a fastener 112 (shown in FIG. 2) having a head can be located with all of the head or a portion of the head beneath a surface of the seat back 104 (e.g., the fastener 112 can be countersunk). Attachment methods for each of the inspection seat 102 components will be further described below.

The seat back 104 can also define several through holes 114 to enable air flow communication between one side of the seat back 104 and another side of the seat back 104 as will be described below. Any suitable number of through holes 114 can be defined by the seat back 104 such that an air flow carrying a scent can pass through the through holes 114 enabling detection of the scent on either side of the seat back 104.

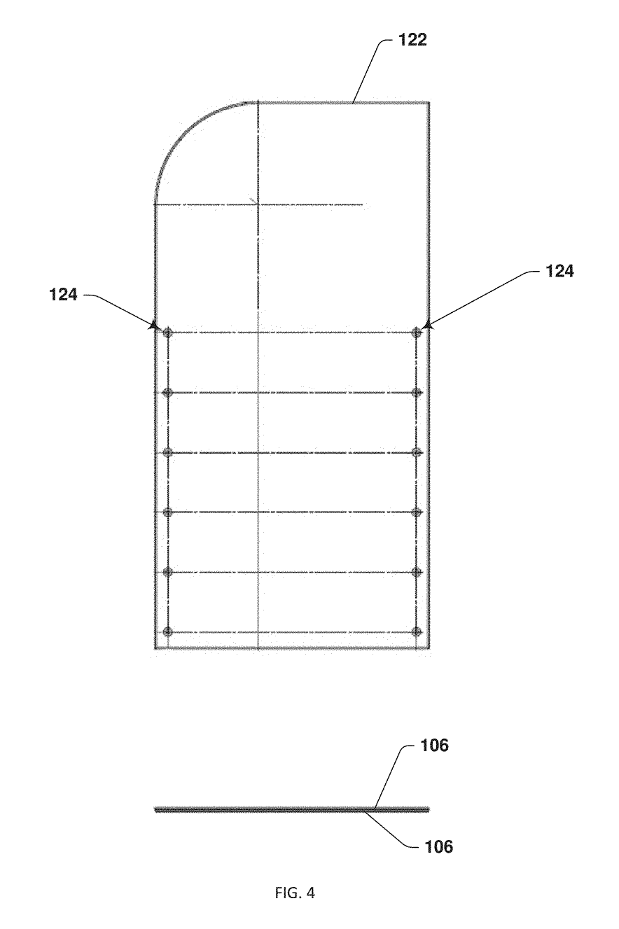

Returning to FIG. 2, the inspection seat 102 also includes a seat of seat sides 116. The set of seat sides 116 includes a seat left side 118 attached to the seat back 104, and a seat right side 122 attached to the seat back 104. Referring to FIG. 4, a detail view of the seat right side 122 is shown. Similar to the seat back 104, the seat right side 122 can be constructed of any suitable material that can meet the expected performance characteristics of the inspection seat 102. In an example, the seat right side 122 comprises an aluminum alloy (e.g., aluminum 6061). In an example, the seat right side 122 comprises an aluminum alloy with a coating 106 (e.g., a black powder coating).

In an example, the seat left side 118 can be identical to the seat right side 122, such that one production part can serve as both the seat left side 118 and the seat right side 122, saving cost, design time, etc. In other examples, the seat left side 118 is a mirror image of the seat right side 122. In an example, the seat left side 118 cannot be identical to the seat right side 122 because of the orientation of the through holes and countersink features of those holes that are presently described.

The seat left side 118 and the seat right side 122 can also define several through holes 124 to facilitate mounting structure that will be further described below. In an example, the several through holes 124 can also include countersunk portions to enable a fastener 112 (shown in FIG. 2) having a head to be located with all of the head or a portion of the head to be located beneath a surface of the seat left side 118 or the seat right side 122 (e.g., the fastener 112 can be countersunk).

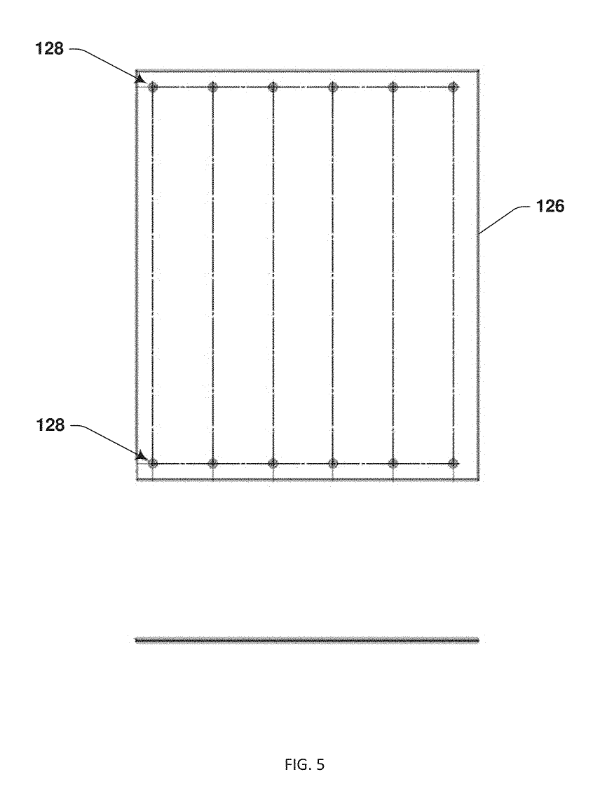

Returning to FIG. 2, the inspection seat 102 also includes a seat front 126. The seat front can be attached to and extends between the seat left side 118 and the seat right side 122. Referring to FIG. 5, a detail view of the seat front 126 is shown. Similar to the seat back 104, the seat left side 118, and the seat right side 122, the seat front 126 can be constructed of any suitable material that can meet the expected performance characteristics of the inspection seat 102. In an example, the seat front 126 comprises an aluminum alloy (e.g., aluminum 6061). In an example, the seat front 126 comprises an aluminum alloy with a coating 106 (e.g., a black powder coating).

The seat front 126 can also define several through holes 128 to facilitate mounting structure that will be further described below. In an example, the several through holes 128 can also include countersunk portions to enable a fastener 112 (shown in FIG. 2) having a head to be located with all of the head or a portion of the head to be located beneath a surface of the seat front 126 (e.g., the fastener 112 can be countersunk).

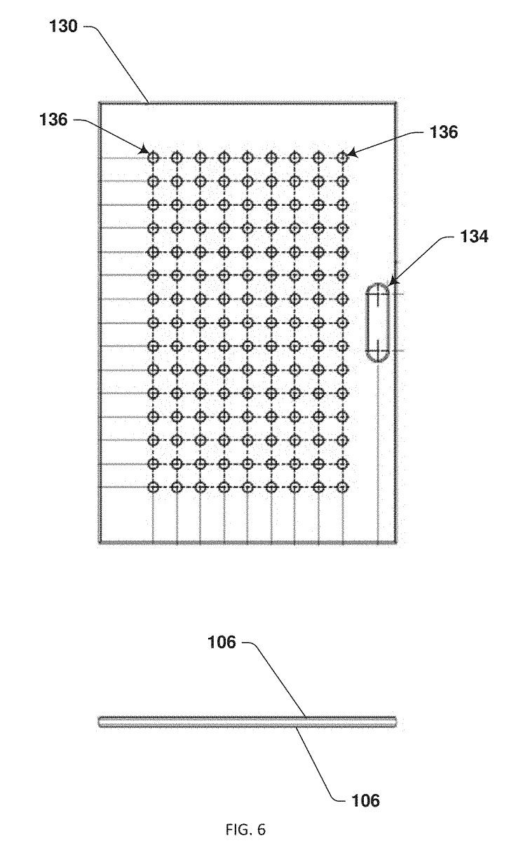

Returning to FIG. 2, the inspection seat 102 also includes a seat top 130 attached to one or the other of the set of seat sides 116 and the seat back 104. The seat top 130 forms the generally horizontal surface upon which a person sits for detection of contraband. In an example, the seat top 130 can simply rest upon the attachment structures that will be described below. Alternatively, one or more “D” rings can be used to hold the seat top 130 in place and snug the seat top 130 to the seat front 126. Referring to FIG. 6, a detail view of the seat top 130 is shown. Similar to the seat back 104, the seat left side 118, the seat right side 122, and the seat front 126, the seat top 130 can be constructed of any suitable material that can meet the expected performance characteristics of the inspection seat 102. In an example, the seat top 130 comprises an aluminum alloy (e.g., aluminum 6061). In an example, the seat top 130 comprises an aluminum alloy with a coating 106 (e.g., a black powder coating).

As shown in the example, the seat top 130 can define an aperture 134 that can be used as a handle to better hold and maneuver the seat top 130. Similarly, the other components of the inspection seat 102 can define an aperture similar to aperture 134 that can be used for handling of the individual components.

The seat top 130 also defines a top aperture 136 to enable air flow communication between a space above the seat top 130 and a lower volume and another side of the seat back 104 as will be described below. Any suitable number of top apertures 136 can be defined by the seat top 130 such that an air flow carrying a scent can pass through the top apertures 136 enabling detection of the scent on either side of the seat top 136.

Returning to FIG. 2, the assembly of the set of seat sides 116, the seat front 126, and the seat top 130 at least partially encloses or defines a lower volume 138 below the seat top 130. In an example, the seat back 104 further defines the lower volume 138, however, this is not necessary. As described previously, the rear aperture 108 of the seat back 104 and the top apertures 136 of the seat top 130 enable air flow communication between a space exterior to the lower volume 139 and the lower volume 138. In an example, the optional through holes 110 of the seat back 104 can also enable air flow communication between the space above the seat top 130 and the space exterior to the lower volume 139 (e.g., the space behind the seat back).

The shown examples illustrate the rear aperture 108 being the sole aperture permitting air flow communication between the lower volume 138 and the space exterior to the lower volume 139 (e.g., the space behind the seat back).

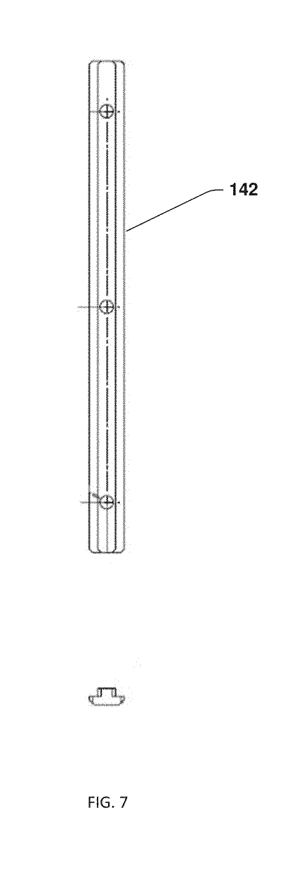

Referring to FIG. 7, a detail view of a seat key 142 is shown. The seat key 142 can be attached to the seat back 104 to begin a process of assembly of the inspection seat 102. The seat key 142 can be constructed of any suitable material including, but not limited to, 1018 steel with a black oxide coating. The seat key 142 can include a “T-shaped” cross-section configured to cooperate with another mounting member that will be described below. The lower portion of the T of the seat key 142 is oriented toward an interior surface of the seat back 104 and arranged such that the seat key 142 has its longest dimension extending in an up/down orientation. In other words, the seat key 142 is approximately vertical when mounted to the seat back 104. Any suitable mounting method is acceptable, including, but not limited to threaded fasteners passing through the through holes 110. In the shown example, eight seat keys 142 are mounted to the seat back 104, however, this number can vary, so long as the seat back 104 is reliably attached to the set of seat sides 116 in the final assembly.

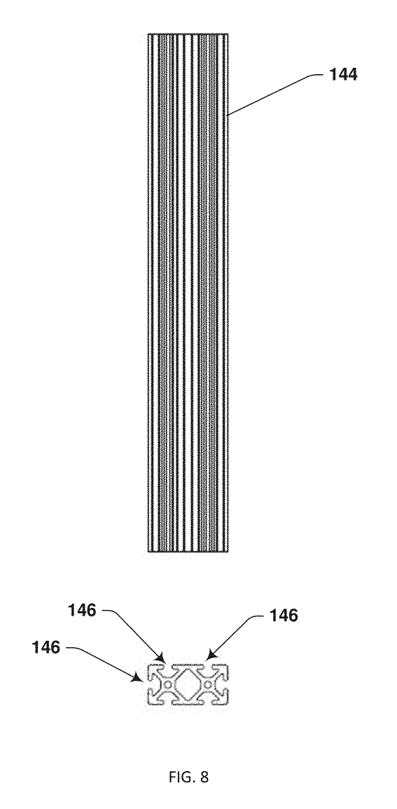

Referring to FIG. 8, a detail view of a rear mounting bar 144 is shown. The rear mounting bar 144 defines a number of slots 146 that are configured to cooperate with the top portion of the T-shaped seat key 142. The rear mounting bar 144 can be constructed of any suitable material including, but not limited to an extrusion of aluminum alloy 8020-1530.

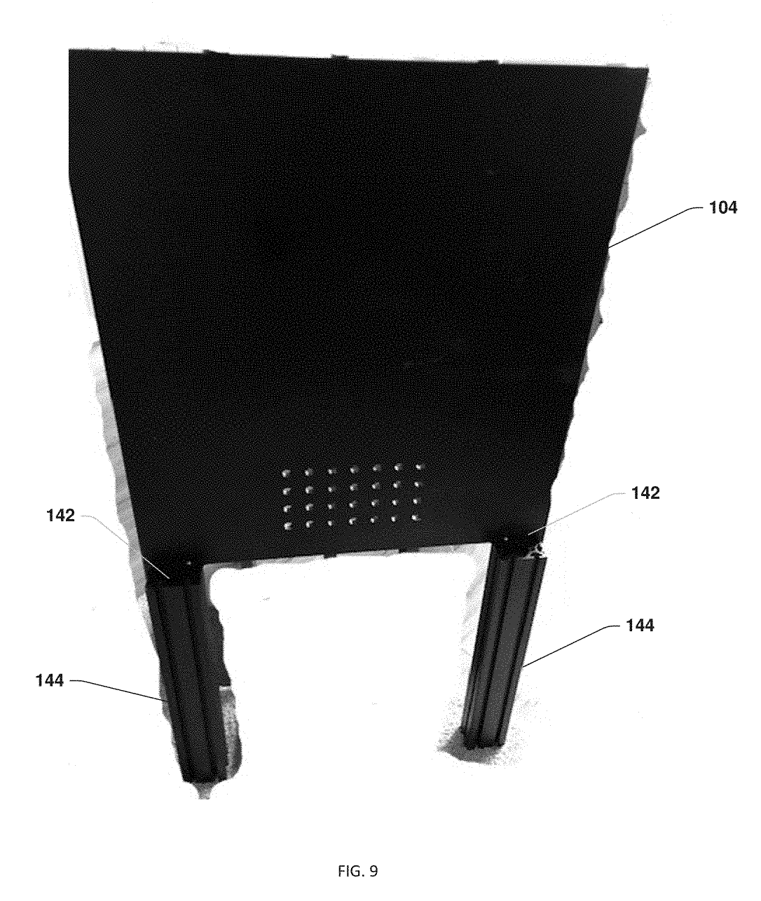

Referring to FIG. 9, one or more rear mounting bars 144 can be slid over the seat keys 142 after they are mounted to the seat back 104. Again, the seat key 142 and the rear mounting bars 144 are oriented in an approximately vertical position such that many of the remainder of the inspection seat 102 components can be selectively placed into sliding engagement with the other components. In an example, the fasteners 112 (shown in FIG. 2) used to secure the seat keys 142 and the rear mounting bars 144 to the seat back 104 can require a specialized tool for installation and removal. Use of the special tool can reduce and/or eliminate the possibility of unauthorized disassembly of the inspection seat 102. Furthermore, the epoxy powder coat for the seat back 104 can be applied after the rear mounting bars 144 have been secured to the seat back 104, thereby “sealing” the rear mounting bars 144 to the seat back 104 in a permanent position. The epoxy powder coat can also at least partially fill any depression in the head of the fastener 112 that cooperates with the specialized tool. The sealing effect and the filling of the depression can both further reduce the possibility of unauthorized tampering with the hardware and the inspection seat 102. Still further, a substance such as Permatex or Loctite can be used on the threaded fastener 112 to make the threaded connection with the rear mounting bars 144 permanent.

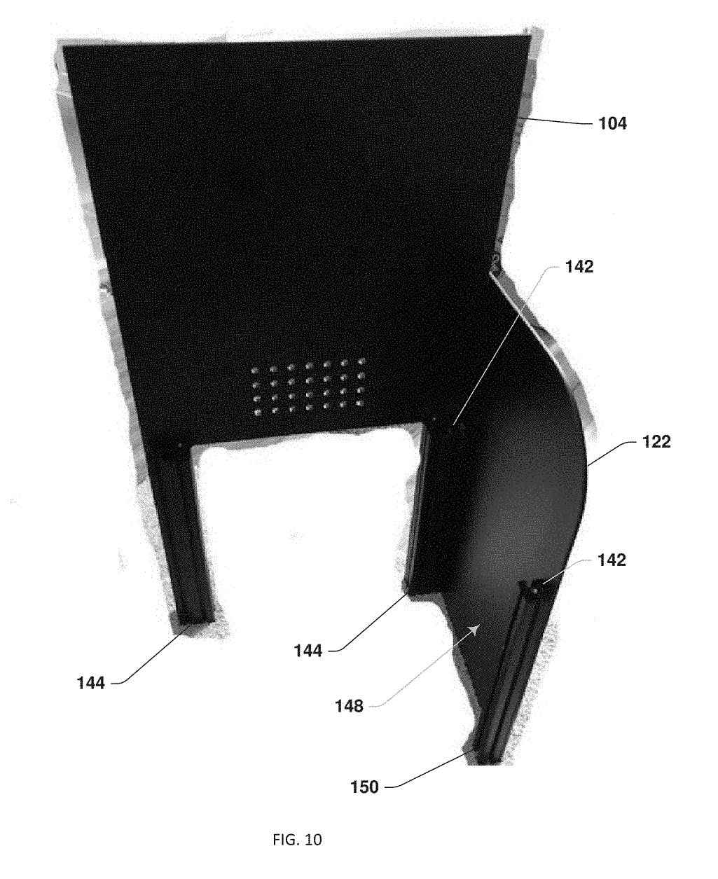

Referring to FIG. 10, the seat right side 122 is then placed in sliding engagement with the seat back 104. This is accomplished by attaching two seat keys 142 to the seat right side 122; one relatively close to the rear of the seat right side 122 on an interior surface 148 and one relatively close to the front of the seat right side 122 on the interior surface 148. The seat key 142 mounted relatively close to the rear of the seat right side 122 cooperates with the rear mounting bar 144 previously described. The seat key 142 mounted relatively close to the front of the seat right side 122 cooperates with a front mounting bar 150.

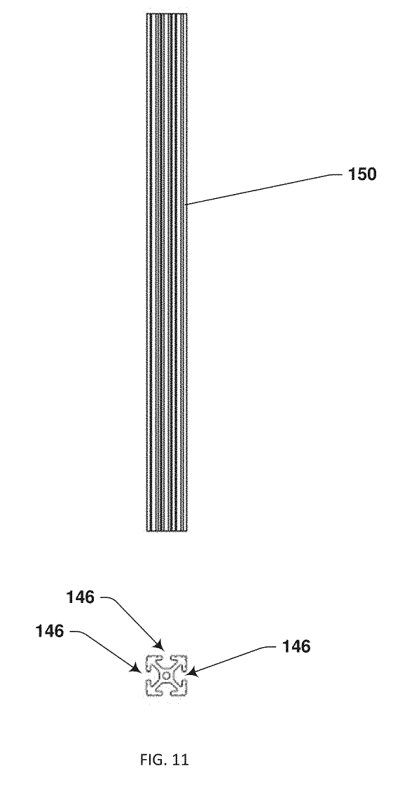

Referring to FIG. 11, a detail view of a front mounting bar 150 is shown. The front mounting bar 150 defines a number of slots 146 that are configured to cooperate with the top portion of the T-shaped seat key 142. The front mounting bar 150 can be constructed of any suitable material including, but not limited to an extrusion of aluminum alloy 8020-1530.

Returning to FIG. 10, the front mounting bar 150 is placed into sliding engagement with the seat key 142 mounted relatively close to the front of the seat right side 122. The front mounting bar 150 can be attached and “sealed” to the seat right side 122 in the same way as the rear mounting bar 144 is attached and “sealed” to the seat back 104 as described above.

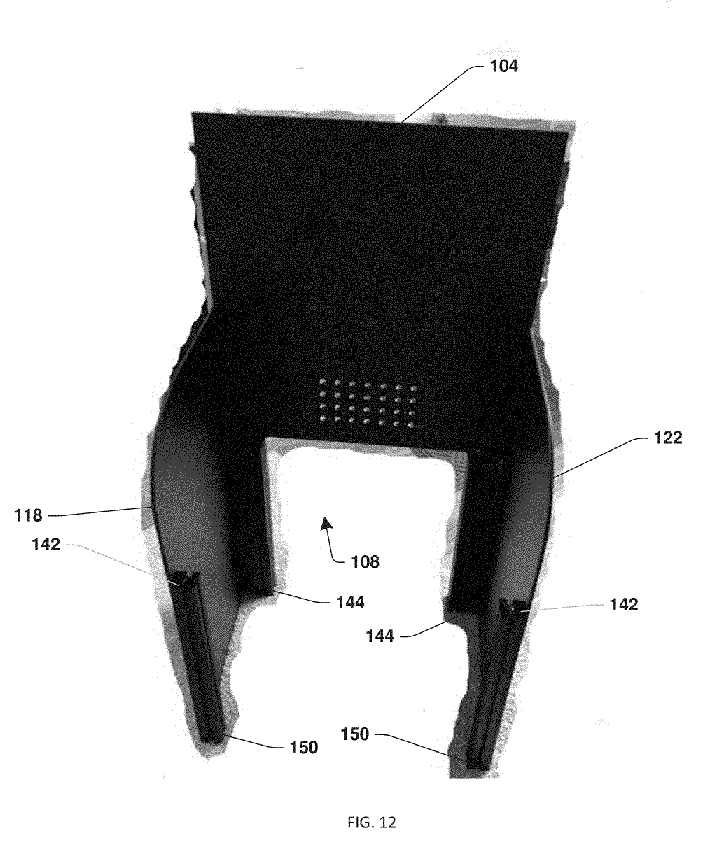

Referring to FIG. 12, the seat left side 118 is then placed into sliding engagement with the seat back 104 through the same process as was described above for the seat right side 122 attachment to the seat back 104. Similarly, a seat key 142 and a front mounting bar 150 are mounted relatively close to the front of the seat left side 118.

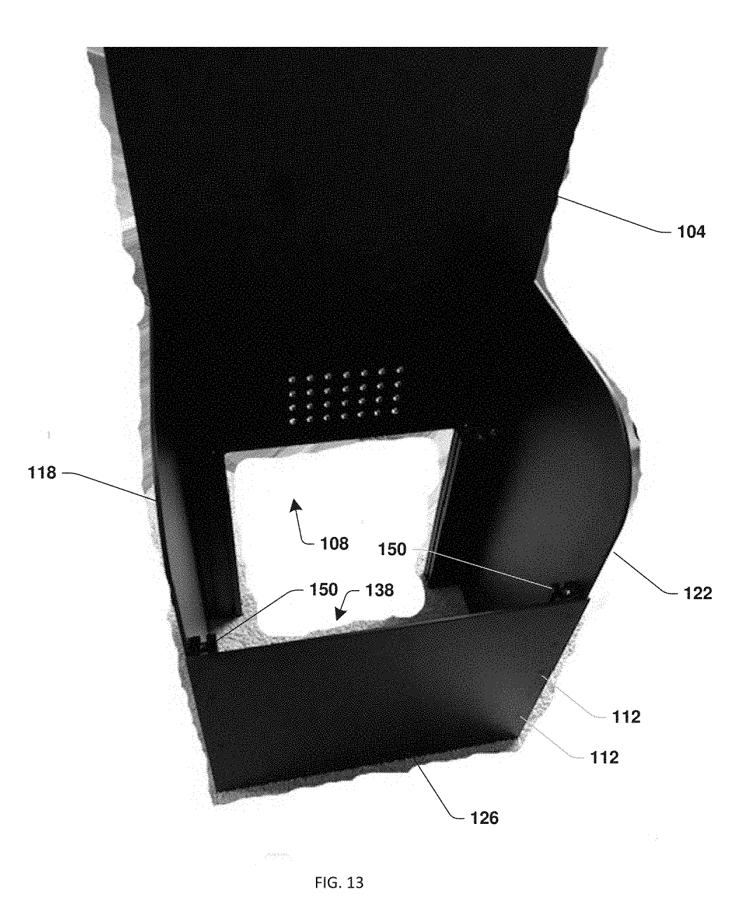

Referring to FIG. 13, the seat front 126 is then attached to the seat left side 118 and the seat right side 122. The sliding engagement of the seat front 126 to the seat left side 118 and the seat right side 122 is similar to the sliding engagement of the components previously described. If so desired, the engagement of the seat back 104, the seat front 126, the seat left side 118 and the seat right side 122 can be further tightened, if necessary, using threaded fasteners 112 (shown in FIG. 2). In an example, the threaded fasteners 112 pass through the through holes 110 and into the seat keys 142. The threaded fasteners can be flathead screws that require a special tool to tighten/loosen, such as a hex wrench, Allen wrench, etc. The special tool requirement can lessen the chance for unauthorized disassembly or vandalism or theft of the inspection seat 102.

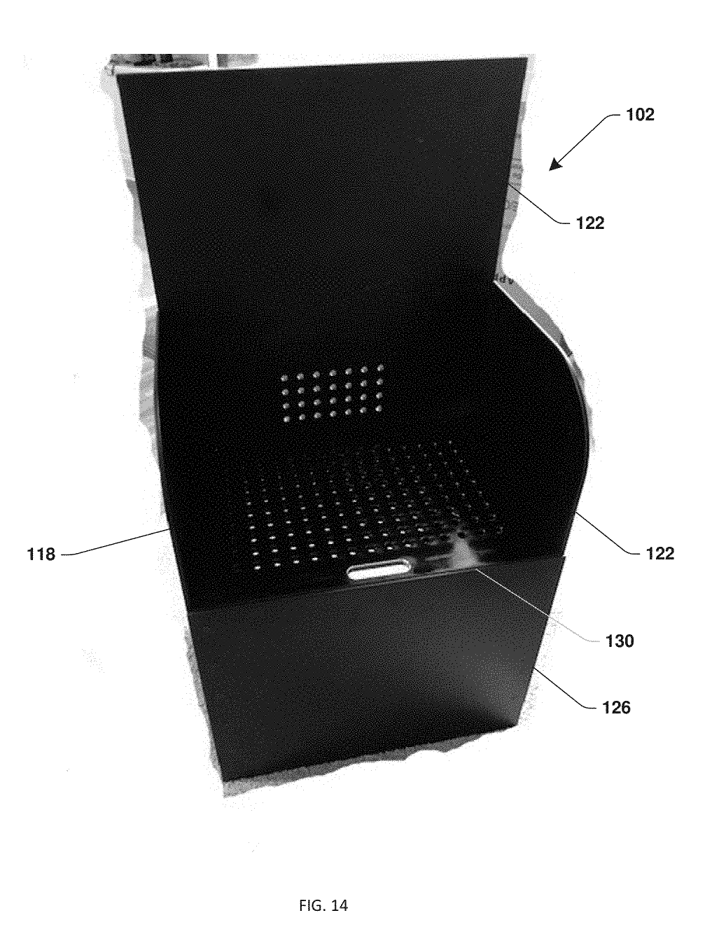

Also shown in FIG. 13 is the lower volume 138 and the rear aperture 108. The final step of the assembly is to place the seat top 130 onto the assembly as shown in FIG. 14. In an example, the corners of the seat top 130 can be placed onto the upper ends of the rear mounting bars 144 and the front mounting bars 150 (shown in FIG. 12). If so desired, the seat top 130 can be fastened to the remainder of the assembly, but this is not necessary. The inspection seat 102 is then ready for use. In summary, the seat back 104, the set of seat sides 116, the and the seat front 126 are selectively attached to one another through sliding engagement to form the inspection seat 102.

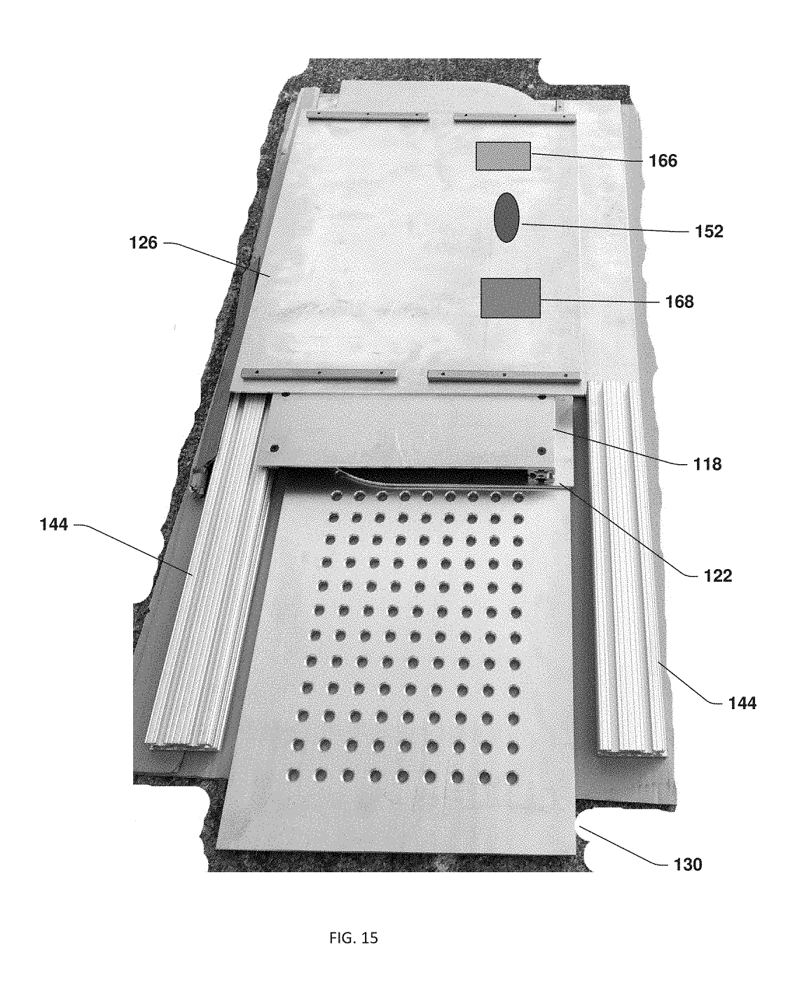

At the time of a desired disassembly process, the above described process is simply reversed. However, it may not be necessary to entirely remove the seat keys 142 from the various inspection seat 102 components. Instead, the fasteners can be loosened and the various seat components can be raised upward to slide the T-shaped portions of the seat keys 142 out of the slots 146 of the rear and front mounting bars 144, 150. Additionally, the seat left side 118 and the seat right side 122 can be generally planar such that the components of the inspection seat 102 can be placed in a relatively small container. In an example, the term relatively small can mean that one dimension (e.g., height) of the container can be significantly less than the other two dimensions of the container. In other words, the inspection seat 102 can be disassembled into its base components to be placed in a relatively flat box that can be stacked or packed to put several disassembled inspection seats 102 into a relatively small volume as shown in FIG. 15.

Additionally, the feature of the seat left side 118 and the seat right side 122 being generally planar can enable the inspection seat abut a flat surface, such as a wall. In an example, the inspection seat 102 cooperates with an adjoining associated inspection seat such that the inspection seat 102 and the associated inspection seat are connected side by side. In this way, the inspection seats 102 can facilitate a relatively quick pass by one canine and handler to detect contraband in a straight-line row of seats.

In an example, the associated inspection seat does not use one of the seat left side 118 and the seat right side 122, such that the cooperation of the inspection seat 102 with the associated inspection seat incorporates two of one of the seat left side 118 and the seat right side 122 and one of the other of the seat left side 118 and the seat right side 122. In other words, it is anticipated that a grouping of two side-by-side inspection seats 102 can share the seat side 118, 122 between them. However, it is to be appreciated that having each inspection seat 102 using an individual seat side 118, 122 can improve modularity to move the seats about as needed and help prevent cross-contamination from one inspection seat 102 to another inspection seat.

Also as shown in FIG. 15, various devices can be added to the inspection seat 102 components such as the seat back 104, the seat left side 118, the seat right side 122, and the seat front 126 to facilitate carrying the components, disassembling the components, and assembling the components. FIG. 15 shows a schematic representation of a knob 152 attached to the seat front 126. In an example, the knob 152 is attached to the interior side of the seat front 126 such that the knob 152 does not interfere with expected operation of the inspection seat 102. Similar devices can be added to the other components as desired.

Returning to FIG. 1, n an example, the inspection seat 102 can cooperate with a wall 160 having a first side 162 and a second side 164, such that the lower volume 138 is accessible from the first side 162 of the wall and the space above the seat top 130 is accessible from the second side 164 of the wall 160. This feature can further enhance the separation of the canine or other detection animal and the person(s) undergoing inspection for contraband.

As will be described below, the rear aperture 108 of the seat back 104 is typically designed such that a scent detection trained animal (e.g., a canine) can place its head or snout within the lower volume 138 to detect contraband being held or hidden by a person sitting in the inspection seat 102. However, other examples can include an air analysis device 166 (schematically shown in FIG. 15) that can be mounted within the lower volume 138. In the event of a situation when a detection canine may not be available, the same inspection seat 102 can be used with the air analysis device 166 to evaluate the presence of contraband on a person or thing located on the seat top 130.

While not necessary, an air movement device 168 (schematically shown in FIG. 15) can also be included in the inspection seat 102 assembly in order to move air between the space above the seat top 130 and the lower volume 138. The automated air movement can then aid the canine or the air analysis device 166 to process a particular amount of air for scents within a particular amount of time. The air movement device 168 can be connected to an electrical source such as a battery by internal or external wires or can be connect to a conventional power outlet. The battery can optionally be rechargeable.

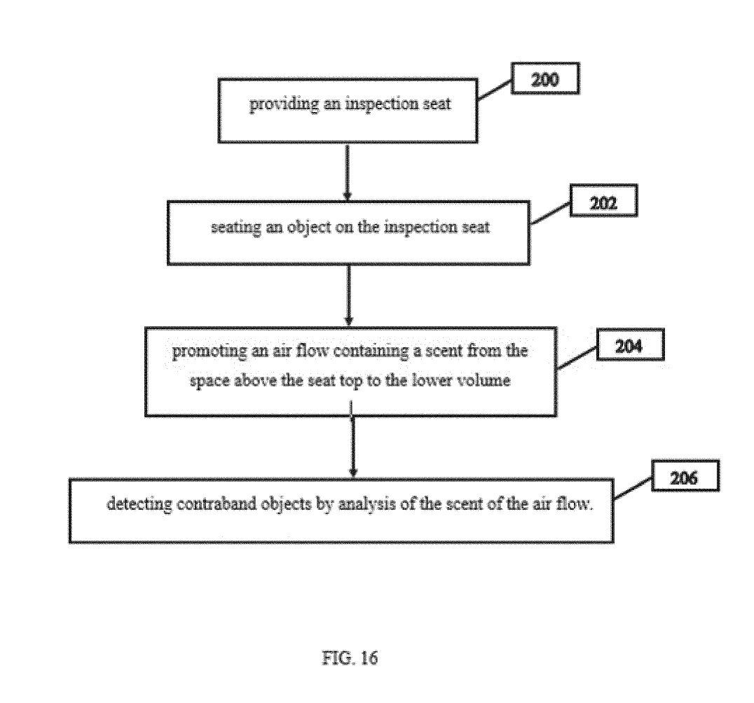

Referring to FIG. 16, an example method of locating contraband with use of the described inspection seat will now be described. The method includes providing an inspection seat 200 as described in the above description. After assembly of the inspection seat, the method includes seating or placing an object on the inspection seat 202. In an example, the object is a person that may be scheduled to enter a relatively secure area. The relatively secure area can be any number of places such as a prison, penitentiary, court house, airport terminal, sporting arena, outdoor or indoor concert venue, a school, etc. In an example, the object may be an inanimate object such as a backpack, box, etc. that may be passing through a relatively secure barrier and the inanimate object may be property of a person passing through the same barrier.

The method also includes promoting an air flow containing a scent from the space above the seat top to the lower volume 204. Promotion of the airflow can be completed via any number of methodologies, and any suitable methodology is satisfactory. In an example, the act of inhaling of an animal trained in the detection of contraband is satisfactory. Other examples can include natural drafts moving between the space above the seat top and the lower volume. Another example includes the previously described air movement device that can include a fan, etc.

The method also includes detecting contraband objects by analysis of the scent of the air flow that has passed from the space above the seat top to the lower volume 206. As noted previously, detecting contraband objects by analysis of the scent of the air flow can include an animal breathing in a portion of the air flow to detect the scent and providing an alert if warranted. In an example, detecting contraband objects can be conducted with the use of an air analysis device that can detect airborne presence of particular elements, molecules, compounds, etc. that indicate the presence of contraband materials.

The canine can be trained to give an indication of contraband detection by either moving its body (sitting for example), or by making some other type of indication. Generally, when contraband detection is made, the operator can optionally move away from the locus with the dog, make sure the lower volume is pulling in only clean air at that point, wait until the canine has stopped responding, and then return to the suspect area. If the dog gives a second indication of a hit or detection, an operator or other personnel can search for the actual contraband. This is only one example of the search and detection process; other examples are contemplated.

In an example, as subjects (e.g., people) are allowed to individually sit in the inspection seat, the airflow passing through the top aperture entrains scents emitted by the subjects and any packages or baggage they may be carrying. The entrained scents are then carried to the lower volume for inhalation and analysis/sensing by the canine. If the canine senses the presence of target scent for which the canine has been trained, the canine will respond with a trained signal to alert a handler that a specific scent has been identified. The canine can be rewarded and the handler, or other security officers, may then direct the interception of the subject or group of subjects who emitted the detected scent.

Use of the described inspection seat and the described methods can provide multiple benefits. The inspection seat can provide desired protection for the animal (e.g., canine) trained in detection of certain types of contraband. The protection for the animal is provided by physical separation of the human subject and the canine as the canine approaches the proper scent detection distance from behind a seated subject. Additionally, the physical barrier provided by the inspection seat between the subject and the canine can eliminate potential scenarios when the subject could initiate unwanted contact with the canine. Additionally, the person and/or people who are being searched can be further protected. In an example, trained canines may not search a person directly due to safety concerns for the canine and the suspected person being searched. The inspection seat can provide privacy for the subject during the search. The person may sit on the inspection seat/bench and may be fully protected from being in contact with the canine so as not to cause any accidental injuries to the person being searched.

Another potential benefit is to pick up air from the immediate vicinity of the target person and move that air to the animal's nose in such a way that the search area can be closer to the height of a canine's head, and done in a way that the canine can move around freely. The term dog or canine is used in this disclosure to represent any animal that can be trained and used to respond to scent. While canines may be the most common animals that can be used in this application, and are preferred because of their intelligence and ability to be trained, any other animal that can be trained to respond may be used and will be included under the name canine or dog in the present application.

Another potential benefit can be a barrier to help protect the canine from accidental exposure to contraband material. The inspection seat may be made from different types of material that may not absorb odor or residue and can be easily sanitized to keep from cross-contamination. An example would be any type of metal or hard plastic. The inspection seat can have a high back and high sides with an opening in the rear of the inspection seat, as described above. In an example, the rear aperture in the inspection seat gives access for the canine to detect contraband on the subject person. The contraband can include, but is not limited to, drugs, drug paraphernalia, explosive materials, explosive materials, etc. The chairs can be set up side by side to make a bench style chair. This may provide faster processing time for multiple people. In an example, dimensions of the chair may be 46 inches by 24 inches by 20 inches to the inspection seat.

Another benefit to the described apparatus and methods is that the inspection seat can be relatively easily disassembled, its parts packed relatively tightly together, transported in a relatively small volume, and reassembled in another location as needed. For example, a security detail may have to process subjects (e.g., people) relatively infrequently at various locations. As such, the inspection seats can be taken to a desired location where the parts can be slidingly engaged with each other and the fasteners can be tightened as needed/desired. The assembly and disassembly process can be relatively quick and can be relatively simple. In an example, the inspection seats can be utilized at a concert, where the inspection seats would not be required to be at that location all the time. The operators/security personnel can transport the needed number of inspection seats to the location, assemble the inspection seats, inspect concert-goers for contraband, disassemble the inspection seats, and then transport them to another location where they are needed or a storage location.

Another benefit can be that a relatively large number of subjects (people) can be searched for contraband within a relatively short amount of time. This can be particularly beneficial at security bottle neck areas such as airport security, concert venue entries, etc. where, at times, large groups of people arrive at one time and desire to pass to a relatively more secure area.

Although the subject matter has been described in language specific to structural features or methodological acts, it is to be understood that the subject matter defined in the appended claims is not necessarily limited to the specific features or acts described above. Rather, the specific features and acts described above are disclosed as example forms of implementing at least some of the claims.

Various operations of embodiments are provided herein. The order in which some or all of the operations described should not be construed to imply that these operations are necessarily order dependent. Alternative ordering will be appreciated having the benefit of this description. Further, it will be understood that not all operations are necessarily present in each embodiment provided herein. Also, it will be understood that not all operations are necessary in some embodiments.

Many modifications may be made to the instant disclosure without departing from the scope or spirit of the claimed subject matter. Unless specified otherwise, “first,” “second,” or the like are not intended to imply a temporal aspect, a spatial aspect, an ordering, etc. Rather, such terms are merely used as identifiers, names, etc. for features, elements, items, etc. For example, a first location and a second location correspond to location A and location B or two different or two identical locations or the same location.

Moreover, “exemplary” is used herein to mean serving as an example, instance, illustration, etc., and not necessarily as advantageous. As used in this application, “or” is intended to mean an inclusive “or” rather than an exclusive “or”. In addition, “a” and “an” as used in this application are to be construed to mean “one or more” unless specified otherwise or clear from context to be directed to a singular form. Also, at least one of A and B or the like means A or B or both A and B. Furthermore, to the extent that “includes”, “having”, “has”, “with”, or variants thereof are used in either the detailed description or the claims, such terms are intended to be inclusive in a manner similar to “comprising”.

Also, although the disclosure has been illustrated and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure. In addition, while a particular feature of the disclosure may have been disclosed with respect to only one of several implementations, such feature may be combined with one or more other features of the other implementations as may be desired and advantageous for any given or particular application.

What is claimed is:

1. An inspection seat comprising:
a seat back, wherein the seat back defines a rear aperture;
a set of seat sides comprising:
a seat left side attached to the seat back;
a seat right side attached to the seat back;
a seat top attached to one or the other of the set of seat sides and the seat back, wherein the seat top defines a top aperture; and
a seat front attached to the set of seat sides,
wherein the seat top, the set of seat sides, and the seat front define a lower volume below the seat top and the rear aperture enables air flow communication between a space exterior to the lower volume and the lower volume,
wherein the top aperture enables air flow communication between a space above the seat top and the lower volume.

2. The inspection seat of claim 1, wherein the seat back defines an aperture that enables air flow communication between a volume above the seat top and a space behind the seat back.

3. The inspection seat of claim 1, wherein the set of seat sides defines a side aperture that enables air flow communication between the space exterior to the lower volume and the lower volume.

4. The inspection seat of claim 1, wherein the rear aperture is the sole aperture permitting air flow communication between the lower volume and the space exterior to the lower volume.

5. The inspection seat of claim 1, wherein the seat back further defines the lower volume.

6. The inspection seat of claim 1, wherein the seat left side is generally planar and the seat right side is generally planar such that the inspection seat can abut a flat surface.

7. The inspection seat of claim 1, wherein the inspection seat cooperates with an adjoining associated inspection seat, such that the inspection seat and the associated inspection seat are connected side by side.

8. The inspection seat of claim 7, wherein the associated inspection seat does not use one of the seat left side and the seat right side, such that the cooperation of the inspection seat with the associated inspection seat incorporates two of one of the seat left side and the seat right side and one of the other of the seat left side and the seat right side.

9. The inspection seat of claim 1, wherein a portion of the inspection seat cooperates with a wall having a first side and a second side, such that the lower volume is accessible from the first side of the wall and the space above the seat top is accessible from the second side of the wall.

10. The inspection seat of claim 1, wherein one of the seat back, the set of seat sides, the seat top, and the seat front comprises a metal.

11. The inspection seat of claim 10, wherein the metal is an aluminum alloy.

12. The inspection seat of claim 11, further comprising a coating on the aluminum alloy.

13. The inspection seat of claim 1, further comprising an air movement device to move air between the space above the seat top and the lower volume.

14. The inspection seat of claim 1, further comprising an air analysis device.

15. The inspection seat of claim 14, wherein the air analysis device is mounted within the lower volume.

16. The inspection seat of claim 1, further comprising:
a plurality of seat keys;
a front mounting bar; and
a rear mounting bar;
wherein the plurality of seat keys are attached to each of the seat back, the set of seat sides, the seat top and the seat front to engage the front mounting bar and the rear mounting bar to enable fastening of the seat back, the set of seat sides, and the seat front in a complete inspection seat.

17. The inspection seat of claim 1, wherein the seat back, the set of seat sides, the seat top, and the seat front are selectively attached to one another through sliding engagement to form the inspection seat.

18. A method of locating contraband comprising:
providing an inspection seat comprising:
a seat back, wherein the seat back defines a rear aperture;
a set of seat sides comprising:
a seat left side attached to the seat back;
a seat right side attached to the seat back; and
a seat top attached to one or the other of the set of seat sides and the seat back, wherein the seat top defines a top aperture; and
a seat front,
wherein the seat top, the set of seat sides, and the seat front define a lower volume below the seat top and the rear aperture enables air flow communication between a space exterior to the lower volume and the lower volume,
wherein the top aperture enables air flow communication between a space above the seat top and the lower volume;
seating an object on the inspection seat;
promoting an air flow containing a scent from the space above the seat top to the lower volume;
detecting contraband objects by analysis of the scent of the air flow.

19. The method of claim 18, wherein the object is a person.

20. The method of claim 19, wherein the detecting contraband objects by analysis of the scent of the air flow comprises an animal breathing in a portion of the air flow to detect the scent and providing an alert if warranted.
